(19) United States
(12) Patent Application Publication
(10) Pub. No.: US 2025/0221736 A1
(43) Pub. Date: Jul. 10, 2025

(54) VASCULAR PUNCTURE APPARATUS

(71) Applicant: TERUMO KABUSHIKI KAISHA, Tokyo (JP)

(72) Inventors: Takito INUKAI, Isehara-shi (JP); Yoichiro KUWANO, Princeton, NJ (US); Takumi FUKUDA, Hadano-shi (JP); Kazuki NAKASONE, Fujinomiya-shi (JP)

(73) Assignee: TERUMO KABUSHIKI KAISHA, Tokyo (JP)

(21) Appl. No.: 19/090,233

(22) Filed: Mar. 25, 2025

Related U.S. Application Data
(63) Continuation of application No. PCT/JP2023/034583, filed on Sep. 22, 2023.

(30) Foreign Application Priority Data
Sep. 26, 2022 (JP) 2022-152985

(51) Int. Cl. A61B 17/34

(52) U.S. Cl. CPC A61B 17/3403; A61B 2017/3413

(57) ABSTRACT

A vascular puncture apparatus that controls a puncture device on the basis of information regarding backflow is provided. A vascular puncture apparatus includes a puncture drive unit that punctures a living body with a puncture device, a backflow detection unit that detects backflow from a blood vessel into the puncture device, and a control unit that controls a position of the puncture device by driving the puncture drive unit on the basis of information regarding the backflow detected by the backflow detection unit. When the backflow detection unit detects backflow from the blood vessel into a proximal end side of the inner needle while the puncture drive unit is inserting the puncture device, the control unit stops insertion of the puncture device.

### CROSS-REFERENCES TO RELATED APPLICATIONS

This application is a continuation of International Application No. PCT/JP2023/034583 filed on Sep. 22, 2023, which claims priority to Japanese Application No. 2022-152985 filed on Sep. 26, 2022, the entire content of both of which is incorporated herein by reference.

### TECHNOLOGICAL FIELD

The present disclosure generally relates to a vascular puncture apparatus for puncturing a blood vessel with a needle.

### BACKGROUND DISCUSSION

In order to secure an access site for drug administration or endovascular treatment, vascular puncture for puncturing a human body with an injection needle is performed. Since an operator cannot visually observe a blood vessel from a skin surface in the vascular puncture, the operator estimates a position of the blood vessel on the basis of standard knowledge of how blood vessels run and skills including tactile perception of vascular pulsation. The vascular puncture, however, often fails, causing physical and mental distress to patients.

In order to identify a puncture position, techniques for visualizing a position of a blood vessel, such as near-infrared imaging, ultrasound echography, and photoacoustic imaging, are used. By visualizing a position of a blood vessel, the operator can rather easily determine a puncture position, a puncture angle, a puncture depth, and the like. An apparatus that drives a needle on the basis of obtained information regarding a position of a blood vessel and that automatically performs puncture is also known. As an apparatus that automatically performs puncture, for example, there is an apparatus described in U.S. Pat. No. 9,364,171.

When a blood vessel is punctured with a needle as a puncture device, backflow is caused, in which blood flows toward a proximal end of the needle through a lumen of the needle. When the operator manually performs puncture, the operator checks a state of the puncture by checking a state of backflow. An apparatus that automatically performs puncture, however, does not control a puncture device on the basis of information regarding backflow.

### SUMMARY

A vascular puncture apparatus is disclosed that controls a puncture device on the basis of information regarding backflow.

A vascular puncture apparatus is disclosed that punctures a living body using a puncture device, the vascular puncture apparatus including a puncture drive unit that punctures the living body with the puncture device, a backflow detection unit that detects backflow into the puncture device from a blood vessel, and a control unit that controls a position of the puncture device by driving the puncture drive unit on a basis of information regarding the backflow detected by the backflow detection unit.

A vascular puncture apparatus is disclosed, which is configured to puncture a living body using an inner needle and an outer tube covering the inner needle, the vascular puncture apparatus comprising: a puncture drive unit configured to puncture the living body with a distal end of the inner needle protruding from a distal end of the outer tube, and wherein the inner needle is removable from the outer tube by the puncture drive unit; a backflow detection unit configured to detect backflow into the inner needle from a blood vessel; and a control unit configured to: control a position of the inner needle by driving the puncture drive unit on a basis of information regarding the backflow detected by the backflow detection unit; stop insertion of the puncture device when the backflow detection unit detects backflow from the blood vessel into a proximal end side of the inner needle while the puncture drive unit is inserting the inner needle.

A method is disclosed for puncturing a living body using a puncture device, the method comprising: puncturing, with a puncture drive unit, the living body with the puncture device; detecting, with a backflow detecting unit, backflow into the puncture device from a blood vessel; and controlling, by a control unit, a position of the puncture device by driving the puncture drive unit on a basis of information regarding the backflow detected by the backflow detection unit.

Since the vascular puncture apparatus configured as described above controls the position of the puncture device on the basis of information regarding backflow that occurs with puncture of a blood vessel with the puncture device, the position of the puncture device can be controlled after accurately grasping a puncture state of the blood vessel with the puncture device, and the puncture can be accurately performed.

### BRIEF DESCRIPTION OF THE DRAWINGS

FIG. 1 is a front view of a vascular puncture apparatus according to an embodiment.

FIG. 2 is a perspective view illustrating an internal structure of a puncture drive unit.

FIG. 3 is a configuration diagram of the vascular puncture apparatus.

FIG. 4 is a diagram illustrating a skin contact surface of a probe body, and is a diagram illustrating a positional relationship with an arm from which a cross-sectional image is obtained.

FIG. 5 is a flowchart of puncture using the vascular puncture apparatus.

FIG. 6 is a conceptual diagram of an obtained echogram.

FIG. 7 is a cross-sectional view illustrating a positional relationship between a blood vessel and an imaging unit.

FIGS. 8A-8C are diagrams illustrating a relationship between an inner needle and the blood vessel during insertion.

FIG. 9 is a flowchart according to a first modification of the puncture using the vascular puncture apparatus.

FIG. 10 is a flowchart according to a second modification of the puncture using the vascular puncture apparatus.

FIG. 11 is a graph illustrating a relationship of an estimated amount of backflow with respect to an insertion depth of the puncture device.

FIGS. 12A-12E are diagrams illustrating a relationship between a puncture device including an outer tube and the blood vessel.

FIG. 13 is a flowchart according to a third modification of the puncture using the vascular puncture apparatus.

FIGS. 14A and 14B are diagrams illustrating a relationship between the puncture device and the blood vessel, where FIG. 14A is a diagram illustrating a state in which the inner needle is located inside the blood vessel and the outer tube is located outside the blood vessel, and FIG. 14B is a diagram illustrating a state in which the inner needle is located outside the blood vessel and the outer tube is located inside the blood vessel.

FIG. 15 is a flowchart according to a fourth modification of the puncture using the vascular puncture apparatus.

FIG. 16 is a front view of a guide wire drive device attached to the puncture drive unit.

FIG. 17 is a front view of a vascular puncture apparatus according to a modification.

### DETAILED DESCRIPTION

Set forth below with reference to the accompanying drawings is a detailed description of embodiments of a vascular puncture apparatus for puncturing a blood vessel with a needle. Note that dimensional ratios in the drawings might be exaggerated for convenience of description and differ from actual ratios.

A vascular puncture apparatus 10 according to an embodiment of the present disclosure is used when puncturing an arm of a human body, and obtains a cross-sectional image of the arm, detects a position of a blood vessel, determines a puncture timing and puncture parameters, and punctures the blood vessel with a needle.

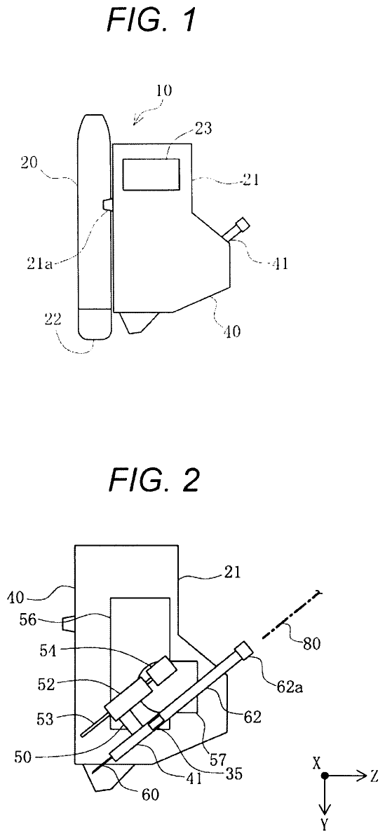

As illustrated in FIG. 1, the vascular puncture apparatus 10 includes a probe body 20 including an imaging unit 22 that comes into contact with a skin surface and that obtains a cross-sectional image of a human body and a puncture drive unit 21 that holds a puncture device 41 and that performs puncture by driving the puncture device 41. The puncture drive unit 21 has a fixing portion 21a engaged with and fixed to the probe body 20, and the probe body 20 and the puncture drive unit 21 are integrated together.

As illustrated in FIG. 2, the puncture drive unit 21 includes a main body 40 that holds the puncture device 41. The main body 40 includes a notification unit 23 for notifying an operator of a puncture state and the like. The notification unit 23 can be configured by a display or the like that displays information. The notification unit 23, however, may be a simpler display unit such as an LED, instead. Alternatively, the notification unit 23 may perform the notification using sound.

The puncture device 41 includes an inner needle 60 that is a hollow needle and that punctures a blood vessel and a hub 62 provided on a proximal end side of the inner needle 60. The inner needle 60 has a distal end opened, and a proximal end of the inner needle 60 communicates with a lumen of the hub 62. The hub 62 includes, at a proximal end of the hub 62, an insertion portion 62a protruding from the main body 40. The insertion portion 62a communicates with the lumen of the hub 62, and a guide wire 80 can be inserted. The guide wire 80 inserted from the insertion portion 62a can be inserted into the inner needle 60 toward a distal end of the inner needle 60 through the lumen of the hub 62 and the inside of the inner needle 60.

The hub 62 is provided with a backflow detection unit 35 that detects backflow from a blood vessel toward a proximal end of the inner needle 60. The backflow detection unit 35 is a sensor capable of detecting a flow of blood in the lumen of the hub 62 from a distal end toward the proximal end of the hub 62, and can be configured by a camera. The backflow detection unit 35 can be configured by a pressure sensor, a proximity sensor, or the like instead of a camera. In addition, the backflow detection unit 35 may be fixed to the main body 40.

The puncture drive unit 21 includes a horizontal direction drive unit 56 supported by the main body 40, a biaxial drive unit 57 supported by the horizontal direction drive unit 56, a puncture direction drive unit 54 supported by the biaxial drive unit 57, a puncture direction guide unit 53 supported by the puncture direction drive unit 54, and a puncture direction movement unit 52 supported by the puncture direction guide unit 53 and movable in a puncture direction. FIG. 2 illustrates X, Y, and Z directions. The X direction corresponds to a width direction of the arm, the Y direction corresponds to a depth direction of the arm, and the Z direction corresponds to a length direction of the arm. The puncture device 41 is fixed to a holding unit 50 provided for the puncture direction movement unit 52.

The biaxial drive unit 57 allows the puncture device 41 to change orientation thereof relative to an X-Z plane. As a result, the orientation of the puncture device 41 with respect to a planar direction of the skin surface can be changed, and the puncture device 41 can enter a blood vessel 100 straight. The horizontal direction drive unit 56 allows the puncture device 41 to move in the X direction. As a result, the puncture device 41 can be moved in the planar direction of the skin surface, and the puncture device 41 can be arranged in such a way as to be able to enter the blood vessel 100. The biaxial drive unit 57 allows the puncture device 41 to change inclination of the puncture device 41 in a Y-Z plane. As a result, an entry angle of the puncture device 41 with respect to the skin surface can be changed.

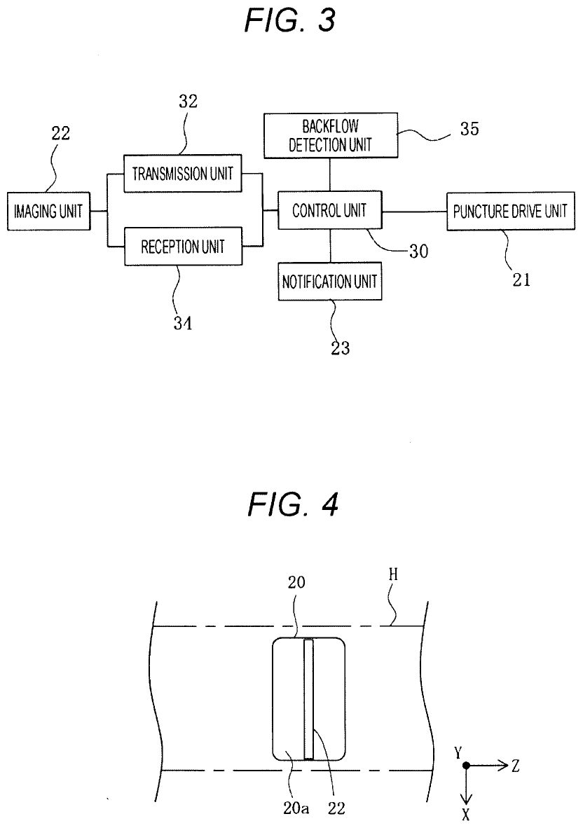

As illustrated in FIG. 3, the vascular puncture apparatus 10 includes the imaging unit 22 that comes into contact with the skin surface and that obtains a cross-sectional image of the human body and a control unit 30 that detects a position of a blood vessel from the cross-sectional image and that controls a position of the puncture device 41 by driving the puncture drive unit 21. The control unit 30 is connected to the imaging unit 22 via a transmission unit 32 and a reception unit 34, can cause the imaging unit 22 to obtain a cross-sectional image, and can receive the obtained cross-sectional image. The control unit 30 is connected to the backflow detection unit 35, and can obtain information regarding backflow detected by the backflow detection unit 35. The control unit 30 is also connected to the notification unit 23 provided for the main body 40, and can cause the notification unit 23 to perform the notification.

As illustrated in FIG. 4, the imaging unit 22 of the probe body 20 is provided in such a way as to extend, in one direction, over substantially entire width of a skin contact surface 20a of the probe body 20 at a central portion of the skin contact surface 20a. The imaging unit 22 is an echograph that includes a transducer which generates an ultrasonic wave and that obtains a cross-sectional image of the inside of the human body by detecting the reflected ultrasonic wave. In the present embodiment, since the imaging unit 22 obtains a cross-sectional image orthogonal to an axial direction of the blood vessel, the imaging unit 22 is arranged such that a length direction of the imaging unit 22 becomes orthogonal to a length direction of an arm H.

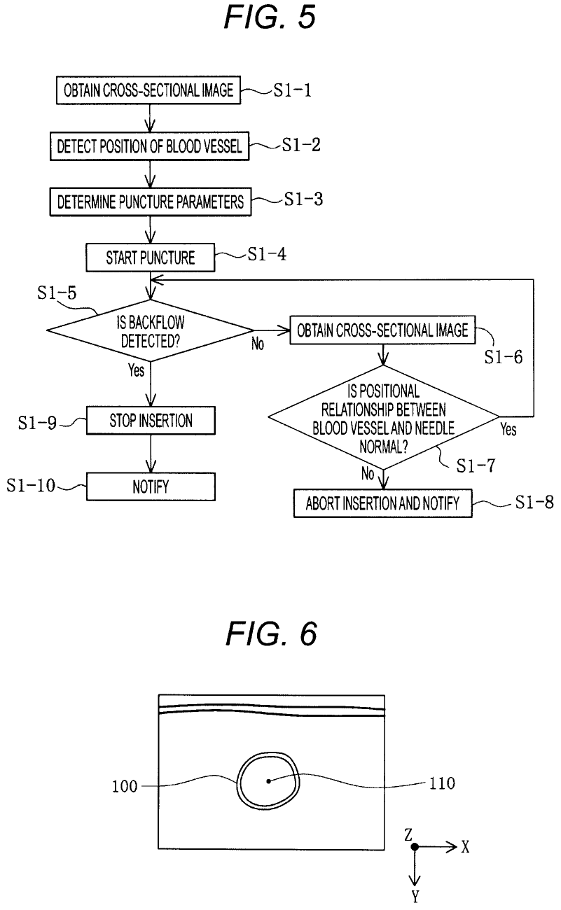

Next, operation of the vascular puncture apparatus 10 will be described along a procedure of puncture. As illustrated in FIG. 5, the control unit 30 obtains a cross-sectional image as illustrated in FIG. 6 from the imaging unit 22 (S1-1). The cross-sectional image includes an image of the blood vessel 100. A horizontal direction in the cross-sectional image is the X direction, a vertical direction in the cross-sectional image is the Y direction, and a direction orthogonal to a paper surface of the cross-sectional image is the Z direction. Coordinates of an upper-left point in the cross-sectional image are defined as a start point (0, 0, 0).

The control unit 30 detects a position of the blood vessel 100 in the obtained cross-sectional image by analyzing the image (S1-2). The control unit 30 detects a region recognized as the blood vessel 100 in the image, and sets a barycentric position 110 of the region as the position of the blood vessel. In order to detect a region recognized as a blood vessel in an image, a large number of images of the same type can be prepared, and a method of machine learning or deep learning can be used. In addition, it is also possible to detect a region with blood flow by a Doppler method in the imaging unit 22 and recognize the region as a region of a blood vessel. The region of the blood vessel needs to be detected from the cross-sectional image while distinguishing an artery and a vein. An artery and a vein can be distinguished on the basis of a position of a bone of the arm H in the cross-sectional image. In addition, when a region with blood flow is detected by the Doppler method, an artery and a vein can be distinguished on the basis of a direction of the blood flow. Coordinates of the detected barycentric position 110 of the blood vessel are defined as (x, y, 0).

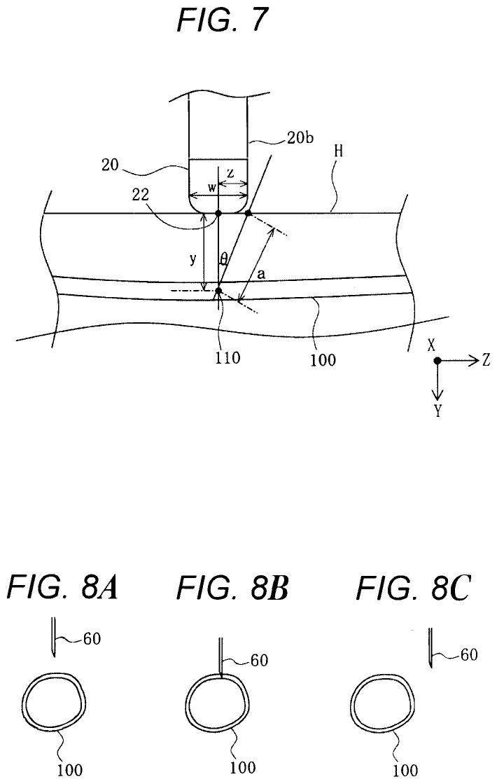

Next, the control unit 30 determines parameters necessary for puncture (S1-3). The parameters necessary for puncture include a puncture direction in the X-Z plane, a puncture position, an entry angle of the puncture device 41, an insertion depth of the puncture device 41, and a puncture speed of the puncture device 41. As illustrated in FIG. 7, since the barycentric position 110 of the blood vessel has been detected from the cross-sectional image obtained by the imaging unit 22, the puncture direction in the X-Z plane is determined such that the puncture device 41 of the puncture drive unit 21 fixed to the probe body 20 is directed to the barycentric position 110.

The puncture position is a position immediately below a side surface 20b of the probe body 20. In this case, a coordinate z of the side surface 20b of the probe body 20 in the Z direction, which is the puncture position, is half a width W of the probe body 20, and is therefore calculated as z=W/2. The puncture angle is an angle θ of a line from the barycentric position of the blood vessel to the puncture position relative to a perpendicular line of the skin surface, and is calculated as θ=arctan (z/y). The puncture depth a is calculated as a=y/cos·θ. The coordinate of the puncture position in the x direction and the puncture depth a of the puncture device 41 are thus defined.

As the parameters of puncture, other parameters may also be used. For example, a puncture start time, a puncture end time, puncture acceleration at a start of puncture, puncture acceleration at an end of puncture, and the like can be used.

After determining the parameters of puncture, the control unit 30 operates the puncture drive unit 21 in accordance with the parameters of puncture to start puncture (S1-4).

The control unit 30 determines whether or not the backflow detection unit 35 detects backflow when the puncture drive unit 21 is inserting the puncture device 41 (S1-5). If the backflow detection unit 35 does not detect backflow, the control unit 30 obtains a cross-sectional image using the imaging unit 22 (S1-6), and determines, on the basis of the obtained cross-sectional image, whether or not a positional relationship between the inner needle 60 and the blood vessel 100 is normal (S1-7). If the inner needle 60 is directed toward the blood vessel 100 in the obtained cross-sectional image as illustrated in FIGS. 8A and 8B, it is determined that the positional relationship between the inner needle 60 and the blood vessel 100 is normal. If the inner needle 60 is not directed toward the blood vessel 100 as illustrated in FIG. 8C, it is determined that the positional relationship between the inner needle 60 and the blood vessel 100 is not normal. The determination of the positional relationship between the inner needle 60 and the blood vessel 100 can be performed by detecting, in the obtained cross-sectional image through an image analysis, coordinates of the distal end of the inner needle 60 and a coordinate range in which the blood vessel 100 is present. Alternatively, the positional relationship between the inner needle 60 and the blood vessel 100 may be determined by a method of machine learning or deep learning.

If it is determined in S1-7 that the positional relationship between the inner needle 60 and the blood vessel 100 is normal, the process returns to step S1-5. If it is determined in S1-7 that the positional relationship between the inner needle 60 and the blood vessel 100 is not normal, the control unit 30 aborts the insertion of the puncture device 41, and the notification unit 23 notifies of the abortion (S1-8).

If the backflow detection unit 35 detects backflow in S1-5, the control unit 30 stops the insertion of the puncture device 41 (S1-9), and the notification unit 23 notifies of the stop (S1-10). When the operator recognizes, on the notification unit 23, that the insertion of the puncture device 41 has been stopped, the operator inserts the guide wire 80 from the hub 62 of the puncture device 41.

By stopping the insertion of the puncture device 41 on the basis of the detection of backflow, the puncture device 41 can thus reliably puncture the blood vessel.

Next, a first modification of the puncture using the vascular puncture apparatus 10 will be described. Because a flow (S1-1 to S1-3) until the puncture parameters are determined in this example is the same as that in FIG. 5, description of the flow (S1-1 to S1-3) until the puncture parameters are determined is omitted.

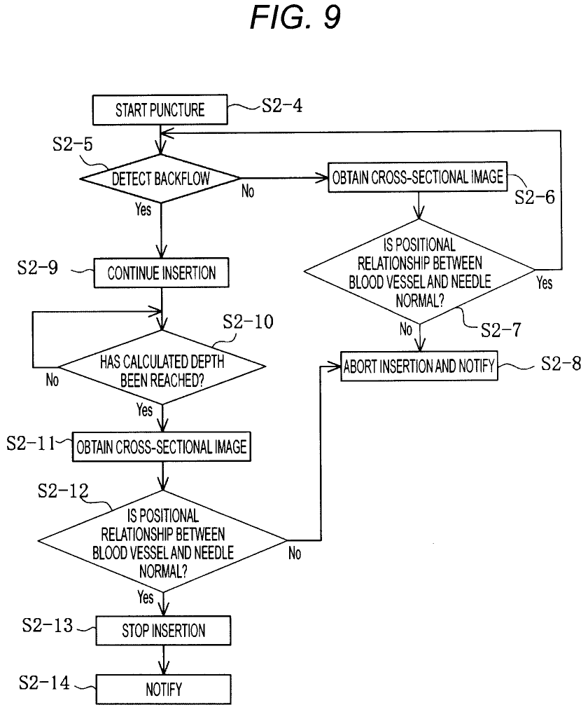

In FIG. 9, the control unit 30 starts puncture with the puncture device 41 (S2-4). The control unit 30 determines whether or not the backflow detection unit 35 detects backflow when the puncture drive unit 21 is inserting the puncture device 41 (S2-5). If the backflow detection unit 35 does not detect backflow, the control unit 30 obtains a cross-sectional image using the imaging unit 22 (S2-6), and determines, on the basis of the obtained cross-sectional image, whether or not the positional relationship between the inner needle 60 and the blood vessel 100 is normal (S2-7). If the positional relationship between the inner needle 60 and the blood vessel 100 is not normal, the control unit 30 aborts the insertion of the puncture device 41, and the notification unit 23 notifies of the abortion (S2-8). If the insertion of the puncture device 41 is aborted in S2-8, the control unit 30 may perform control in such a way as to remove the puncture device 41.

If the backflow detection unit 35 detects backflow in S2-5, the control unit 30 continues the insertion of the puncture device 41 (S2-9). The control unit 30 determines whether or not the insertion depth of the puncture device 41 has reached a calculated depth while the puncture device 41 is being inserted (S2-10). When the insertion depth of the puncture device 41 reaches the calculated depth, the control unit 30 obtains a cross-sectional image using the imaging unit 22 (S2-11), and determines, on the basis of the obtained cross-sectional image, whether or not the positional relationship between the inner needle 60 and the blood vessel 100 is normal (S2-12). If the positional relationship between the inner needle 60 and the blood vessel 100 is normal, the control unit 30 stops the insertion of the puncture device 41 (S2-13), and causes the notification unit 23 to notify of the stop (S2-14). If the positional relationship between the inner needle 60 and the blood vessel 100 is not normal in S2-12, the control unit 30 performs the abortion processing in S2-8.

The insertion of the puncture device 41 is thus continued at a time of the detection of the backflow, and the puncture device 41 is inserted to the calculated depth of the puncture parameters and then stopped, so that the puncture device 41 is inserted deeper into the blood vessel without being immediately stopped after penetrating a vessel wall. In a state immediately after the puncture device 41 penetrates the vessel wall, there is a possibility that the inner needle 60 has not sufficiently stuck in the blood vessel, but by inserting the puncture device 41 to the deeper into the blood vessel, the inner needle 60 can more reliably puncture the blood vessel.

Next, a second modification of the puncture using the vascular puncture apparatus 10 will be described. Because a flow (S1-1 to S1-3) until the puncture parameters are determined in this example is the same as that in FIG. 5, description of the flow (S1-1 to S1-3) until the puncture parameters are determined is omitted.

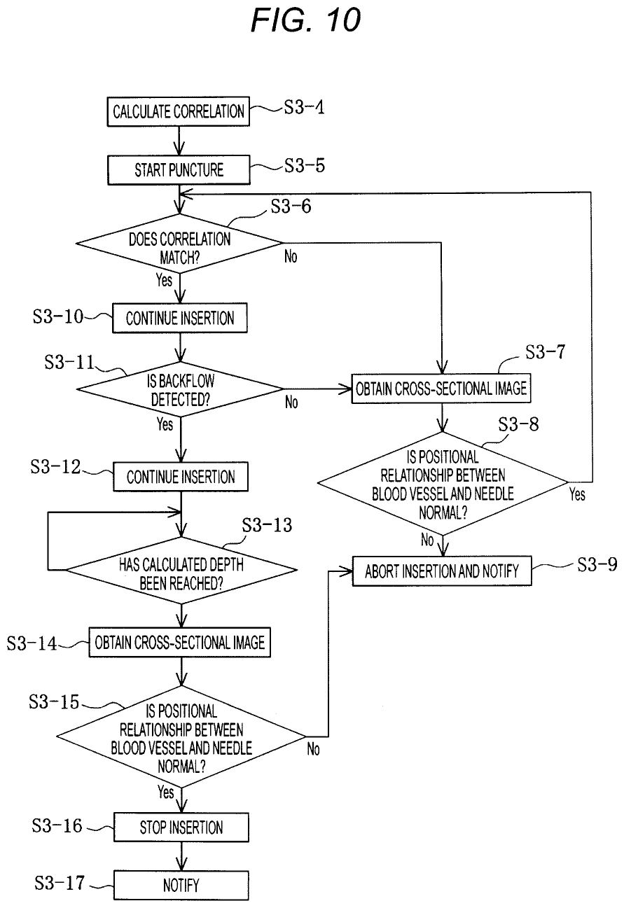

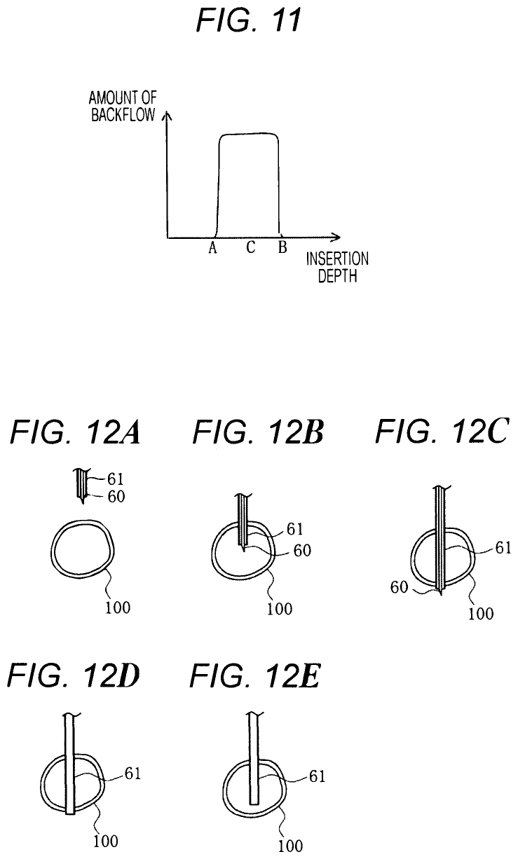

In FIG. 10, after determining the puncture parameters, the control unit 30 calculates a correlation of an estimated amount of backflow with respect to the insertion depth of the puncture device 41 on the basis of the cross-sectional image obtained by the imaging unit 22 (S3-4). Since the backflow occurs from a depth A at which the distal end of the inner needle 60 penetrates a vessel wall on a puncture front side to a depth B at which the distal end of the inner needle 60 penetrates a vessel wall on a puncture back side, a relationship as illustrated in FIG. 11 is calculated. In addition, a calculated depth C among the puncture parameters is between the depth A and the depth B.

Next, the control unit 30 starts puncture with the puncture device 41 (S3-5). When the puncture is started, the control unit 30 starts detection of backflow using the backflow detection unit 35. The control unit 30 determines whether or not a state of the detected backflow matches the calculated correlation (S3-6). If the state of the backflow matches the correlation, the insertion of the puncture device 41 is continued (S3-10). If the state of the backflow does not match the correlation, the control unit 30 obtains a cross-sectional image using the imaging unit 22 (S3-7), and determines, on the basis of the obtained cross-sectional image, whether or not the positional relationship between the inner needle 60 and the blood vessel 100 is normal (S3-8). If the positional relationship between the inner needle 60 and the blood vessel 100 is not normal, the control unit 30 aborts the insertion of the puncture device 41, and the notification unit 23 notifies of the abortion (S3-9). If the positional relationship between the inner needle 60 and the blood vessel 100 is normal, the control unit 30 repeats the flow from S3-6.

While the puncture device 41 is being inserted, the control unit 30 determines whether or not the backflow detection unit 35 detects backflow (S3-11). If the backflow detection unit 35 does not detect backflow, the control unit 30 performs the flow of S3-7 to S3-9 described above. If backflow is detected in S3-11, the control unit 30 continues the insertion of the puncture device 41 (S3-12). When the puncture device 41 reaches the calculated depth of the puncture parameters (S3-13), the control unit 30 obtains a cross-sectional image using the imaging unit 22 (S3-14), determines the positional relationship between the inner needle 60 and the blood vessel 100 (S3-15), stops the insertion of the puncture device 41 if the positional relationship is normal (S3-16), and notifies of the stop on the notification unit 23 (S3-17). If determining in S3-15 that the positional relationship between the inner needle 60 and the blood vessel 100 is not normal, the control unit 30 performs the abortion processing in S3-9.

By calculating the correlation of the estimated amount of backflow with respect to the insertion depth of the puncture device 41 in advance and determining whether the state of backflow matches the correlation during the insertion of the puncture device 41, the puncture position of the inner needle 60 can thus be more accurately grasped, and accuracy of puncture can be increased.

Next, a third modification of the puncture using the vascular puncture apparatus 10 will be described. In this example, as illustrated in FIG. 12A, the puncture device 41 includes an inner needle 60 and an outer tube 61 covering the inner needle 60, and a distal end of the inner needle 60 protrudes from a distal end of the outer tube 61. In addition, the inner needle 60 can be moved to a proximal end of the outer tube 61 by the puncture drive unit 21 and removed. The backflow detection unit 35 detects backflow between the inner needle 60 and the outer tube 61.

When the puncture device 41 provided with the outer tube 61 is inserted into the blood vessel 100, both the inner needle 60 and the outer tube 61 enter the blood vessel as illustrated in FIG. 12B. At this time, backflow occurs in the inner needle 60 and between the inner needle 60 and the outer tube 61.

When the puncture device 41 is further inserted, both the inner needle 60 and the outer tube 61 penetrate vessel walls of the blood vessel on a side of the insertion and an opposite side as illustrated in FIG. 12C. At this time, since the distal end of the inner needle 60 and a distal end of the outer tube 61 are located outside the blood vessel, backflow does not occur.

As illustrated in FIG. 12D, when the inner needle 60 and the outer tube 61 penetrate the blood vessel, the inner needle 60 is removed. Furthermore, as illustrated in FIG. 12E, by moving the remaining outer tube 61 to a proximal end side, the distal end of the outer tube 61 is located in the blood vessel, and the guide wire 80 can be inserted.

A flow of puncture in this example will be described. Because a flow (S1-1 to S1-3) until the puncture parameters are determined in this example is the same as that in FIG. 5, description of the flow (S1-1 to S1-3) until the puncture parameters is omitted.

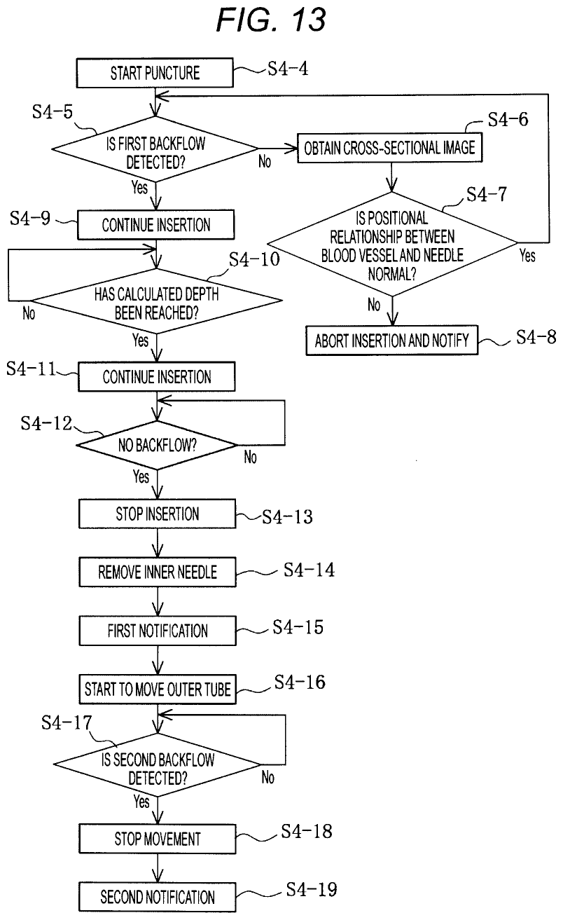

In FIG. 13, when the puncture of the puncture device 41 is started (S4-4), the control unit 30 determines whether or not the backflow detection unit 35 detects a first backflow while the puncture drive unit 21 is inserting the puncture device 41 (S4-5). The first backflow is a backflow that first occurs after the puncture device 41 starts puncture, and is a backflow that occurs in a state of FIG. 12B. If the backflow detection unit 35 does not detect backflow, the control unit 30 obtains a cross-sectional image using the imaging unit 22 (S4-6), and determines, on the basis of the obtained cross-sectional image, whether or not the positional relationship between the inner needle 60 and the blood vessel 100 is normal (S4-7). If the positional relationship between the inner needle 60 and the blood vessel 100 is not normal, the control unit 30 aborts the insertion of the puncture device 41, and the notification unit 23 notifies of the abortion (S4-8).

If the backflow detection unit 35 detects the first backflow in S4-5, the control unit 30 continues the insertion of the puncture device 41 (S4-9). The control unit 30 determines whether or not the insertion depth of the puncture device 41 has reached the calculated depth while the puncture device 41 is being inserted (S4-10). Here, since the puncture device 41 is once inserted to a position penetrating the blood vessel as illustrated in FIG. 12C, when the insertion depth of the puncture device 41 reaches the calculated depth, the control unit 30 further continues the insertion of the puncture device 41 (S4-11).

The control unit 30 continues the insertion of the puncture device 41 until the backflow detection unit 35 no longer detects the backflow (S4-12). When the backflow of the puncture device 41 is no longer detected, the control unit 30 stops the insertion while assuming that the puncture device 41 has penetrated the blood vessel (S4-13).

Next, the control unit 30 removes the inner needle 60 from the outer tube 61 (S4-14), and causes the notification unit 23 to make a first notification about the removal (S4-15). Subsequently, the control unit 30 starts to move the outer tube 61 to the proximal end side (S4-16). The control unit 30 determines whether or not the backflow detection unit 35 detects a second backflow while the outer tube 61 is being moved (S4-17). The second backflow is a backflow that occurs again after backflow stops once, and is a backflow that occurs in a state of FIG. 12E. The second backflow has a larger amount or a higher rate than the first backflow, since the inner needle 60 has been removed.

The backflow detection unit 35 has a configuration capable of detecting the amount or rate of backflow. For this purpose, the hub 62 is provided with a measuring tube with a scale branching from the lumen, and the backflow detection unit 35 including a camera can detect the amount of backflow by detecting a division at which blood flowing into the measuring tube reaches. Alternatively, the backflow detection unit 35 may include a flow meter in the lumen of the hub 62.

When the backflow detection unit 35 detects, in S4-17, the second backflow having a larger amount or a higher rate than the first backflow, the control unit 30 stops the movement of the outer tube 61 (S4-18) and causes the notification unit 23 to make a second notification about the stop (S4-19).

The puncture device 41 provided with the outer tube 61, too, can thus control insertion into a blood vessel by detecting the first backflow and the second backflow.

The backflow detection unit 35 may be configured to be able to detect backflow into the proximal end side of the inner needle 60 in addition to backflow between the inner needle 60 and the outer tube 61. In this case, the backflow detection unit 35 includes a sensor such as a camera in each of the outer tube 61 and the inner needle 60 or a tubular body communicating with the outer tube 61 and the inner needle 60.

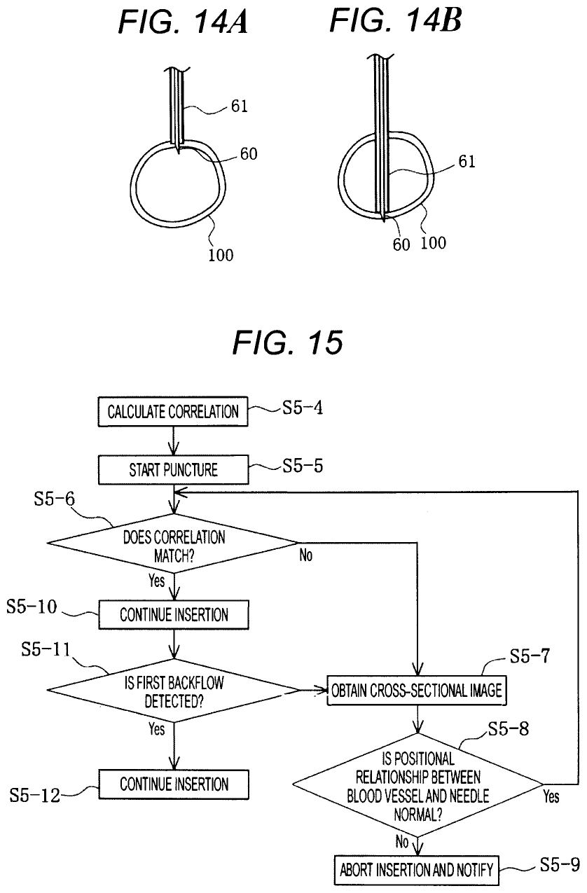

As illustrated in FIG. 14A, in a state in which only the inner needle 60 protruding from the outer tube 61 has entered the blood vessel 100 but the outer tube 61 has not reached the blood vessel 100, backflow into the proximal end side of the inner needle 60 occurs, but backflow does not occur between the inner needle 60 and the outer tube 61. When the backflow detection unit 35 detects this state, it can be determined that the puncture device 41 is in a state immediately before reaching the blood vessel 100.

As illustrated in FIG. 14B, in a state in which only the inner needle 60 protruding from the outer tube 61 has penetrated the blood vessel 100 but the outer tube 61 is located in the blood vessel 100, backflow occurs between the inner needle 60 and the outer tube 61, but backflow into the proximal end side of the inner needle 60 does not occur. When the backflow detection unit 35 detects this state, therefore, it can be determined that the puncture device 41 is in a state immediately before penetrating the blood vessel 100.

When the backflow detection unit 35 is capable of detecting both the backflow between the inner needle 60 and the outer tube 61 and the backflow into the proximal end side of the inner needle 60, accuracy of the detection of the position of the puncture device 41 can be further improved.

Next, a fourth modification of the puncture using the vascular puncture apparatus 10 will be described. Because a flow (S1-1 to S1-3) until the puncture parameters are determined in this example is the same as that in FIG. 5, description of the flow (S1-1 to S1-3) until the puncture parameters are determined is omitted. In the present example, the puncture device 41 includes an inner needle 60 and an outer tube 61.

In FIG. 15, after determining the puncture parameters, the control unit 30 calculates the correlation of the estimated amount of backflow with respect to the insertion depth of the puncture device 41 on the basis of the cross-sectional image obtained by the imaging unit 22 (S5-4). Here, the correlation is calculated for a range from when the puncture device 41 is inserted into the blood vessel 100 to when the puncture device 41 reaches and penetrates the blood vessel 100. That is, the estimated amount of backflow for the first backflow is calculated.

Next, the control unit 30 starts puncture with the puncture device 41 (S5-5). When the puncture is started, the control unit 30 determines whether or not a state of the backflow detected by the backflow detection unit 35 matches the calculated correlation (S5-6). If the state of the backflow matches the correlation, the insertion of the puncture device 41 is continued (S5-10). If the state of the backflow does not match the correlation, the control unit 30 obtains a cross-sectional image using the imaging unit 22 (S5-7), and determines, on the basis of the obtained cross-sectional image, whether or not the positional relationship between the inner needle 60 and the blood vessel 100 is normal (S5-8). If the positional relationship between the inner needle 60 and the blood vessel 100 is not normal, the control unit 30 aborts the insertion of the puncture device 41, and the notification unit 23 notifies of the abortion (S5-9). If the positional relationship between the inner needle 60 and the blood vessel 100 is normal, the control unit 30 repeats the flow from S5-6.

While the puncture device 41 is being inserted, the control unit 30 determines whether or not the backflow detection unit 35 detects the first backflow (S5-11). If the backflow detection unit 35 does not detect backflow, the control unit 30 performs the flow of S5-7 to S5-9 described above. If backflow is detected in S5-11, the control unit 30 continues the insertion of the puncture device 41 (S5-12). Since a flow after S5-12 is the same as the flow after S4-11 in FIG. 13, description of the flow after S5-12 is omitted.

By determining whether a state of backflow matches the correlation of the estimated amount of backflow with respect to the insertion depth at the time of insertion of the puncture device 41 including the inner needle 60 and the outer tube 61, the puncture position of the puncture device 41 can thus be more accurately grasped, and the accuracy of puncture can be increased.

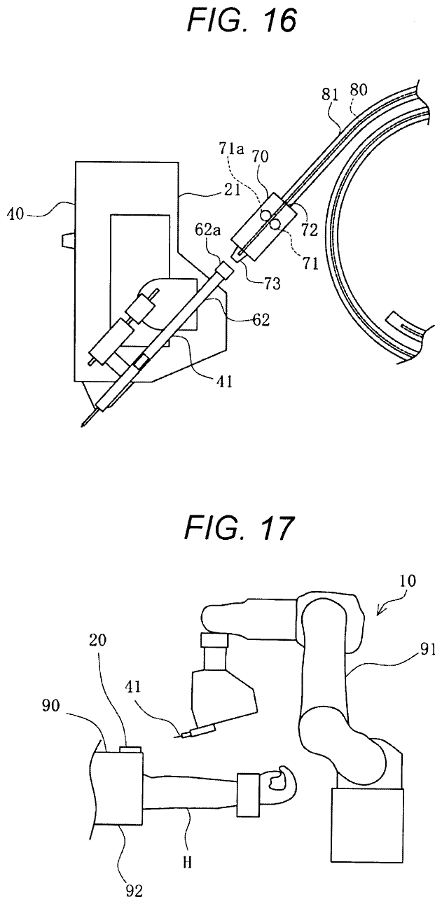

The guide wire 80 is inserted into the puncture device 41 as described above. The guide wire 80 may be manually inserted by the operator, or may be automatically inserted by a guide wire drive device 70. As illustrated in FIG. 16, the guide wire drive device 70 includes a guide wire insertion unit 72 in which an end of a holder tube 81 holding the guide wire 80 is held and into which the guide wire 80 is inserted, a guide wire drive unit 71 that moves the inserted guide wire 80, and a coupling unit 73 coupled to the insertion portion 62a of the hub 62 of the puncture device 41. The guide wire drive unit 71 includes two rollers 71a sandwiching the guide wire 80, and can move the guide wire 80 to both sides in a longitudinal direction.

The guide wire drive device 70 is coupled to the hub 62 after the puncture drive unit 21 causes the puncture device 41 to puncture the blood vessel. The coupling of the guide wire drive device 70 to the hub 62 is performed by the operator, but a coupling device that moves the guide wire drive device 70 and couples the guide wire drive device to the hub 62 may also be provided, which allows the guide wire drive device 70 to be automatically coupled to the puncture device 41.

By providing the guide wire drive device 70 including the guide wire drive unit 71, manual work after the puncture can thus be automated.

The puncture drive unit 21 only needs to be able to perform puncture by driving the puncture device 41, and is not limited to the configuration of the puncture drive unit 21 in the present embodiment. As illustrated in FIG. 17, the vascular puncture apparatus 10 includes a puncture drive unit 91 including a robot arm capable of moving the puncture device 41 in three dimensions and a probe body 20. The puncture drive unit 91 can cause the puncture device 41 to perform puncture from any position at any angle through control based on a sensor that is not illustrated.

When the arm H is inserted into a base 90 and fixed by a fixing portion 92, the probe body 20 obtains a cross-sectional image, and the control unit 30 determines a puncture timing and puncture parameters from the cross-sectional image. The puncture drive unit 91 punctures the arm H with the puncture device 41 in accordance with a determination whether or not the puncture is possible, the position or a direction of the puncture, and the puncture parameters, and controls the puncture by detecting backflow.

As described above, (1) a vascular puncture apparatus 10 according to the present embodiment is a vascular puncture apparatus 10 that punctures a living body with a puncture device 41 and that includes a puncture drive unit 21 which punctures the living body with the puncture device 41, a backflow detection unit 35 which detects backflow from a blood vessel into the puncture device 41, and a control unit 30 which controls a position of the puncture device 41 by driving the puncture drive unit 21 on the basis of information regarding the backflow detected by the backflow detection unit 35. Since the vascular puncture apparatus 10 configured as described above controls the position of the puncture device 41 on the basis of information regarding backflow that occurs with puncture of a blood vessel with the puncture device 41, the position of the puncture device 41 can be controlled after accurately grasping a puncture state of the blood vessel with the puncture device 41, and the puncture can be accurately performed.

(2) In the vascular puncture apparatus 10 according to (1), the puncture device 41 may be configured by an inner needle 60, and the control unit 30 may stop insertion of the puncture device 41 when the backflow detection unit 35 detects backflow from the blood vessel into a proximal end side of the inner needle 60 while the puncture drive unit 21 is inserting the puncture device 41. As a result, since the vascular puncture apparatus 10 can confirm that the puncture device 41 has entered the blood vessel by detecting backflow, it is possible to accurately determine puncture of the blood vessel.

(3) The vascular puncture apparatus 10 according to (1) may further include a probe body 20 including an imaging unit 22 that obtains a cross-sectional image of a human body, in which the puncture device 41 may be configured by an inner needle 60, and the control unit 30 may calculate an estimated insertion depth of the puncture device 41 on a basis of the cross-sectional image obtained by the imaging unit 22, continue insertion of the puncture device 41 when the backflow detection unit 35 detects backflow from the blood vessel into a proximal end side of the inner needle 60 while the puncture drive unit 21 is inserting the puncture device 41, obtain another cross-sectional image using the imaging unit 22 when an insertion depth of the puncture device 41 reaches the estimated calculated depth, and determine, on a basis of the obtained cross-sectional image, whether or not a positional relationship between the inner needle 60 and the blood vessel is normal, and stop the insertion of the puncture device 41 if the positional relationship between the inner needle 60 and the blood vessel is normal, and perform certain abortion processing if the positional relationship between the inner needle 60 and the blood vessel is normal. As a result, since the vascular puncture apparatus 10 can insert the puncture device 41 deeper into the blood vessel than in a state immediately after the puncture device 41 penetrates a vessel wall, it is possible to perform reliable puncture.

(4) The vascular puncture apparatus 10 according to (1) may further include a probe body 20 including an imaging unit 22 that obtains a cross-sectional image of a human body, in which the puncture device 41 may be configured by an inner needle 60, and the control unit 30 may calculate an estimated insertion depth of the puncture device 41 on a basis of the cross-sectional image obtained by the imaging unit 22, and stop insertion of the puncture device 41 when the backflow detection unit 35 detects backflow from the blood vessel into a proximal end side of the inner needle 60 and an insertion depth of the puncture device 41 reaches the estimated calculated depth while the puncture drive unit 21 is inserting the puncture device 41. As a result, since the vascular puncture apparatus 10 can insert the puncture device 41 deeper into the blood vessel than in a state immediately after the puncture device 41 penetrates a vessel wall, it is possible to perform reliable puncture.

(5) The vascular puncture apparatus 10 according to (1) may further include a probe body 20 including an imaging unit 22 that obtains a cross-sectional image of a human body, in which the puncture device 41 may be configured by an inner needle 60, and the control unit 30 may calculate a correlation of an estimated amount of backflow with respect to an insertion depth of the puncture device 41 on a basis of the cross-sectional image obtained by the imaging unit 22 and determine whether or not a state of the backflow detected by the backflow detection unit 35 matches the correlation while the puncture drive unit 21 is inserting the puncture device 41, and if the state of the backflow matches the correlation, continues the insertion of the puncture device 41 and if the state of the backflow does not match the correlation, performs certain abortion processing. As a result, the vascular puncture apparatus 10 can grasp a puncture position of the inner needle 60 more accurately, and the accuracy of puncture can be increased.

(6) In the vascular puncture apparatus 10 according to (1), the puncture device 41 may include an inner needle 60 and an outer tube 61 covering the inner needle 60, a distal end of the inner needle 60 may protrude from a distal end of the outer tube 61, and the inner needle 60 may be removable from the outer tube 61 by the puncture drive unit 21, and the control unit 30 may continue insertion of the puncture device 41 when the backflow detection unit 35 detects a first backflow from the blood vessel between the inner needle 60 and the outer tube 61, while the puncture drive unit 21 is inserting the puncture device 41, stop the insertion of the puncture device 41 when the backflow detection unit 35 detects stop of the first backflow while the puncture drive unit 21 is inserting the puncture device 41 after the detection of the first backflow, remove the inner needle 60 from the outer tube 61 when the insertion of the puncture device 41 is stopped, move the outer tube 61 to a proximal end side when the inner needle 60 is removed from the outer tube 61, and stop the movement of the outer tube 61 when the backflow detection unit 35 detects a second backflow from the blood vessel to the proximal end side of the outer tube 61 while the puncture drive unit 21 is moving the outer tube 61 to the proximal end side. As a result, the vascular puncture apparatus 10 can perform, using the puncture device 41 including the outer tube 61 outside the inner needle 60, puncture in which the blood vessel is penetrated once and then only the outer tube 61 remains in the blood vessel by removing the inner needle 60 while detecting backflow.

(7) In the vascular puncture apparatus 10 according to (1), the puncture device 41 may include an inner needle 60 and an outer tube 61 covering the inner needle 60, a distal end of the inner needle 60 may protrude from a distal end of the outer tube 61, and the inner needle 60 may be removable from the outer tube 61 by the puncture drive unit 21, and the control unit 30 may stop insertion of the puncture device 41 when the backflow detection unit 35 detects a first backflow from the blood vessel between the inner needle 60 and the outer tube 61 and then detects stop of the first backflow while the puncture drive unit 21 is inserting the puncture device 41, and stop movement of the outer tube 61 when the backflow detection unit 35 detects a second backflow from the blood vessel into the proximal end side of the outer tube 61 while the puncture drive unit 21 is moving the outer tube 61 to the proximal end side. As a result, the vascular puncture apparatus 10 can perform, using the puncture device 41 including the outer tube 61 outside the inner needle 60, puncture in which the blood vessel is penetrated once and then only the outer tube 61 remains in the blood vessel by removing the inner needle 60 while detecting backflow.

(8) The vascular puncture apparatus 10 according to any one of (1) to (7) may further include a notification unit 23 controlled by the control unit 30, in which the control unit 30 may cause the notification unit 23 to make a notification if determining, on a basis of information regarding the backflow from the backflow detection unit 35, that a distal end of the puncture device 41 is located in the blood vessel. As a result, the operator can smoothly perform manual work after the puncture.

(9) The vascular puncture apparatus 10 according to any one of (1) to (8) may further include a guide wire drive unit 71 that inserts a guide wire 80 into the puncture device 41, in which the control unit 30 may cause the guide wire drive unit 71 to insert the guide wire 80 into the blood vessel if determining, on a basis of information regarding the backflow from the backflow detection unit 35, that a distal end of the puncture device 41 is located in the blood vessel. As a result, the vascular puncture apparatus 10 can automate the insertion of the guide wire 80 performed after the puncture with the puncture device 41.

Note that the present disclosure is not limited to the embodiment described above, and those skilled in the art can make various modifications within the technical idea of the present disclosure. For example, although a monitor that displays an obtained cross-sectional image is not illustrated in the present embodiment, the vascular puncture apparatus 10 may be connected to the monitor so that the cross-sectional image can be viewed.

The vascular puncture apparatus 10 may be configured such that the puncture drive unit 21, the backflow detection unit 35, and the control unit 30 are wirelessly connected to one another, and information can be transmitted and received.

The detailed description above describes embodiments of a vascular puncture apparatus for puncturing a blood vessel with a needle. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A vascular puncture apparatus configured to puncture a living body using a puncture device, the vascular puncture apparatus comprising:
a puncture drive unit configured to puncture the living body with the puncture device;
a backflow detection unit configured to detect backflow into the puncture device from a blood vessel; and
a control unit configured to control a position of the puncture device by driving the puncture drive unit on a basis of information regarding the backflow detected by the backflow detection unit.

2. The vascular puncture apparatus according to claim 1, wherein
the puncture device includes an inner needle; and
the control unit is configured to stop insertion of the puncture device when the backflow detection unit detects backflow from the blood vessel into a proximal end side of the inner needle while the puncture drive unit is inserting the puncture device.

3. The vascular puncture apparatus according to claim 1, further comprising:
a probe body including an imaging unit, the imaging unit configured to obtain a cross-sectional image of a human body, wherein
the puncture device includes an inner needle; and
the control unit is configured to:
calculate an estimated insertion depth of the puncture device on a basis of the cross-sectional image obtained by the imaging unit;
continue insertion of the puncture device when the backflow detection unit detects backflow from the blood vessel into a proximal end side of the inner needle while the puncture drive unit is inserting the puncture device;
obtain another cross-sectional image using the imaging unit when an insertion depth of the puncture device reaches the estimated calculated depth, and determines, on a basis of the obtained cross-sectional image, whether or not a positional relationship between the inner needle and the blood vessel is normal; and
stop the insertion of the puncture device if the positional relationship between the inner needle and the blood vessel is normal, and perform certain abortion processing if the positional relationship between the inner needle and the blood vessel is normal.

4. The vascular puncture apparatus according to claim 1, further comprising:
a probe body including an imaging unit, the imaging unit configured to obtain a cross-sectional image of a human body, wherein
the puncture device includes an inner needle; and
the control unit is configured to:
calculate an estimated insertion depth of the puncture device on a basis of the cross-sectional image obtained by the imaging unit; and
stop insertion of the puncture device when the backflow detection unit detects backflow from the blood vessel into a proximal end side of the inner needle and an insertion depth of the puncture device reaches the estimated calculated depth while the puncture drive unit is inserting the puncture device.

5. The vascular puncture apparatus according to claim 1, further comprising:
a probe body including an imaging unit, the imaging unit configured to obtain a cross-sectional image of a human body, wherein
the puncture device includes an inner needle; and
the control unit is configured to:
calculate a correlation of an estimated amount of backflow with respect to an insertion depth of the puncture device on a basis of the cross-sectional image obtained by the imaging unit; and
determine whether or not a state of the backflow detected by the backflow detection unit matches the correlation while the puncture drive unit is inserting the puncture device, and if the state of the backflow matches the correlation, continues the insertion of the puncture device and if the state of the backflow does not match the correlation, performs certain abortion processing.

6. The vascular puncture apparatus according to claim 1, wherein
the puncture device includes an inner needle and an outer tube covering the inner needle, a distal end of the inner needle protrudes from a distal end of the outer tube, and the inner needle is removable from the outer tube by the puncture drive unit; and
the control unit is configured to:
continue insertion of the puncture device when the backflow detection unit detects a first backflow from the blood vessel between the inner needle and the outer tube, while the puncture drive unit is inserting the puncture device;
stop the insertion of the puncture device when the backflow detection unit detects stop of the first backflow while the puncture drive unit is inserting the puncture device after the detection of the first backflow;
remove the inner needle from the outer tube when the insertion of the puncture device is stopped;
move the outer tube to a proximal end side when the inner needle is removed from the outer tube; and
stop the movement of the outer tube when the backflow detection unit detects a second backflow from the blood vessel to the proximal end side of the outer tube while the puncture drive unit is moving the outer tube to the proximal end side.

7. The vascular puncture apparatus according to claim 1, wherein
the puncture device includes an inner needle and an outer tube covering the inner needle, a distal end of the inner needle protrudes from a distal end of the outer tube, and the inner needle is removable from the outer tube by the puncture drive unit; and
the control unit is configured to:
stop insertion of the puncture device when the backflow detection unit detects a first backflow from the blood vessel between the inner needle and the outer tube and then detects stop of the first backflow while the puncture drive unit is inserting the puncture device; and
stop movement of the outer tube when the backflow detection unit detects a second backflow from the blood vessel into the proximal end side of the outer tube while the puncture drive unit is moving the outer tube to the proximal end side.

8. The vascular puncture apparatus according to claim 1, further comprising:
a notification unit controlled by the control unit, wherein
the control unit is configured to cause the notification unit to make a notification if determining, on a basis of information regarding the backflow from the backflow detection unit, that a distal end of the puncture device is located in the blood vessel.

9. The vascular puncture apparatus according to claim 1, further comprising:
a guide wire drive unit configured to insert a guide wire into the puncture device; and
wherein the control unit is configured to cause the guide wire drive unit to insert the guide wire into the blood vessel if determining, on a basis of information regarding the backflow from the backflow detection unit, that a distal end of the puncture device is located in the blood vessel.

10. A vascular puncture apparatus configured to puncture a living body using an inner needle and an outer tube covering the inner needle, the vascular puncture apparatus comprising:
a puncture drive unit configured to puncture the living body with a distal end of the inner needle protruding from a distal end of the outer tube, and wherein the inner needle is removable from the outer tube by the puncture drive unit;
a backflow detection unit configured to detect backflow into the inner needle from a blood vessel; and
a control unit configured to:
control a position of the inner needle by driving the puncture drive unit on a basis of information regarding the backflow detected by the backflow detection unit;
stop insertion of the puncture device when the backflow detection unit detects backflow from the blood vessel into a proximal end side of the inner needle while the puncture drive unit is inserting the inner needle.

11. The vascular puncture apparatus according to claim 10, further comprising:
a probe body including an imaging unit, the imaging unit configured to obtain a cross-sectional image of a human body; and
wherein the control unit is configured to:
calculate an estimated insertion depth of the inner needle and the outer tube on a basis of the cross-sectional image obtained by the imaging unit;
continue insertion of the inner needle and the outer tube when the backflow detection unit detects backflow from the blood vessel into a proximal end side of the inner needle while the puncture drive unit is inserting the inner needle and the outer tube;
obtain another cross-sectional image using the imaging unit when an insertion depth of the puncture device reaches the estimated calculated depth, and determines, on a basis of the obtained cross-sectional image, whether or not a positional relationship between the inner needle and the blood vessel is normal; and
stop the insertion of the inner needle and the outer tube if the positional relationship between the inner needle and the blood vessel is normal, and perform certain abortion processing if the positional relationship between the inner needle and the blood vessel is normal.

12. A method for puncturing a living body using a puncture device, the method comprising:
puncturing, with a puncture drive unit, the living body with the puncture device;
detecting, with a backflow detecting unit, backflow into the puncture device from a blood vessel; and
controlling, by a control unit, a position of the puncture device by driving the puncture drive unit on a basis of information regarding the backflow detected by the backflow detection unit.

13. The method according to claim 12, wherein the puncture device includes an inner needle, the method further comprises:
stopping insertion of the puncture device when the backflow detection unit detects backflow from the blood vessel into a proximal end side of the inner needle while the puncture drive unit is inserting the puncture device.

14. The method according to claim 12, wherein the puncture device includes an inner needle, the method further comprises:
obtaining a cross-sectional image of a human body with a probe body including an imaging unit;
calculating, with the control unit, an estimated insertion depth of the puncture device on a basis of the cross-sectional image obtained by the imaging unit;
continuing insertion of the puncture device when the backflow detection unit detects backflow from the blood vessel into a proximal end side of the inner needle while the puncture drive unit is inserting the puncture device;
obtaining another cross-sectional image using the imaging unit when an insertion depth of the puncture device reaches the estimated calculated depth, and determines, on a basis of the obtained cross-sectional image, whether or not a positional relationship between the inner needle and the blood vessel is normal; and
stopping the insertion of the puncture device if the positional relationship between the inner needle and the blood vessel is normal, and perform certain abortion processing if the positional relationship between the inner needle and the blood vessel is normal.

15. The method according to claim 12, wherein the puncture device includes an inner needle, the method further comprises:
obtain a cross-sectional image of a human body with a probe body including an imaging unit;
calculating, with the control unit, an estimated insertion depth of the puncture device on a basis of the cross-sectional image obtained by the imaging unit; and
stopping insertion of the puncture device when the backflow detection unit detects backflow from the blood vessel into a proximal end side of the inner needle and an insertion depth of the puncture device reaches the estimated calculated depth while the puncture drive unit is inserting the puncture device.

16. The method according to claim 12, wherein the puncture device includes an inner needle, the method further comprises:
obtaining a cross-sectional image of a human body with a probe body including an imaging unit;
calculating, with the control unit, a correlation of an estimated amount of backflow with respect to an insertion depth of the puncture device on a basis of the cross-sectional image obtained by the imaging unit; and
determine, with the control unit, whether or not a state of the backflow detected by the backflow detection unit matches the correlation while the puncture drive unit is inserting the puncture device, and if the state of the backflow matches the correlation, continues the insertion of the puncture device and if the state of the backflow does not match the correlation, performs certain abortion processing.

17. The method according to claim 12, wherein the puncture device includes an inner needle and an outer tube covering the inner needle, a distal end of the inner needle protrudes from a distal end of the outer tube, and the inner needle is removable from the outer tube by the puncture drive unit, and the method further comprises:
continuing insertion of the puncture device when the backflow detection unit detects a first backflow from the blood vessel between the inner needle and the outer tube, while the puncture drive unit is inserting the puncture device;
stopping the insertion of the puncture device when the backflow detection unit detects stop of the first backflow while the puncture drive unit is inserting the puncture device after the detection of the first backflow;
removing the inner needle from the outer tube when the insertion of the puncture device is stopped;
moving the outer tube to a proximal end side when the inner needle is removed from the outer tube; and
stopping the movement of the outer tube when the backflow detection unit detects a second backflow from the blood vessel to the proximal end side of the outer tube while the puncture drive unit is moving the outer tube to the proximal end side.

18. The method according to claim 12, wherein the puncture device includes an inner needle and an outer tube covering the inner needle, a distal end of the inner needle protrudes from a distal end of the outer tube, and the inner needle is removable from the outer tube by the puncture drive unit, and the method further comprises:
stopping insertion of the puncture device when the backflow detection unit detects a first backflow from the blood vessel between the inner needle and the outer tube and then detects stop of the first backflow while the puncture drive unit is inserting the puncture device; and
stopping movement of the outer tube when the backflow detection unit detects a second backflow from the blood vessel into the proximal end side of the outer tube while the puncture drive unit is moving the outer tube to the proximal end side.

19. The method according to claim 12, further comprising:
causing a notification unit to make a notification if determining, on a basis of information regarding the backflow from the backflow detection unit, that a distal end of the puncture device is located in the blood vessel.

20. The method according to claim 12, further comprising:
inserting a guide wire into the puncture device; and
causing the guide wire drive unit to insert the guide wire into the blood vessel if determining, on a basis of information regarding the backflow from the backflow detection unit, that a distal end of the puncture device is located in the blood vessel.
